(19) United States
(12) Patent Application Publication
(10) Pub. No.: US 2004/0237971 A1
(43) Pub. Date: Dec. 2, 2004

(54) Methods and apparatuses for controlling optical aberrations to alter modulation transfer functions

(76) Inventors: Hema Radhakrishnan, Cambridge (GB); Daniel O'Leary, Cambridge (GB); Arthur Ho, Clovelly (AU); Brien Holden, Kingsford (AU); Shahina Pardhan, Cambridge (GB); Richard Calver, Cambridge (GB)

Correspondence Address: JOHN S. PRATT, ESQ, KILPATRICK STOCKTON, LLP, 1100 PEACHTREE STREET, ATLANTA, GA, 30309, US

(21) Appl. No.: 10/833,246

(22) Filed: Apr. 27, 2004

Related U.S. Application Data
(60) Provisional application No. 60/475,017, filed on Jun. 2, 2003.

(51) Int. Cl. A61B 19/00; A61B 18/20

(52) U.S. Cl. 128/898; 606/5

(57) ABSTRACT

A method and apparatus are disclosed for controlling optical aberrations to alter modulation transfer functions by providing an ocular system comprising a predetermined corrective factor to produce substantially corrective stimuli for repositioning medium- and high-spatial frequency peaks relative to one another to alter accommodative lag. The invention will be used to provide continuous, useful clear visual images while simultaneously retarding or abating the progression of myopia or hypermetropia.

### CROSS-REFERENCE

This Application claims the benefit of priority of U.S. Provisional Application Serial No. 60/475,017, filed Jun. 2, 2003.

### FIELD OF THE INVENTION

The present invention is directed to methods and apparatuses for retarding or eliminating the progression of myopia in an individual by controlling aberrations, thereby manipulating the positioning of the medium and high spatial frequency peaks of a visual image while simultaneously providing clear imaging.

### BACKGROUND OF THE INVENTION

The prevalence of myopia (short sightedness) is increasing rapidly, especially in Asian children. Studies, for example, have shown a dramatic rise in the incidence of myopia (−0.25 D or more) in 7 year old Taiwanese children, from 4% to 16% between 1986 and 2000, and the prevalence of myopia (−0.25 D or more) in Taiwanese school children aged 16 to 18 years is as high as 84%. A population-based study in mainland China reports that 55% of girls and 37% of boys aged 15 have significant myopia (−1.00 D or more).

Studies show that 50% of people with high myopia (over −6.00 D) have some form of retinal pathology. Myopia significantly increases the risk of retinal detachment, (depending on the level of myopia), posterior cataract and glaucoma. The optical, visual and potential pathological effects of myopia and its consequent inconvenience and cost to the individual and community, makes it desirable to have effective strategies to slow the progress, or prevent or delay the onset of myopia, or limit the amount of myopia occurring in both children and young adults.

Thus, a large percentage of the world's population has myopia at a level that requires some form of optical correction in order to see clearly. It is known that myopia, regardless of age of onset, tends to increase in amount requiring stronger and stronger correction. These corrections are available through a wide range of devices including spectacles, contact lenses and refractive surgery. However, they do little if anything to slow or stop the progression of myopia.

One form of myopia, (often called “congenital myopia”), occurs at birth, is usually of high level, and may become progressively worse. A second type (sometimes called “juvenile myopia” or “school myopia”) begins in children at age 5 to 10 years and progresses through to adulthood or sometimes beyond. A third ‘type’ of myopia (which may be referred to as “adult myopia”) begins in young adulthood or late teenage years (16 to 19 years of age) and increases during adulthood, sometimes leveling off and at other times continuing to increase.

Strategies to prevent or slow myopia have been suggested that involve pharmacological interventions with anti-muscarinic drugs such as atropine (that are usually used to paralyze accommodation), or pirenzipine. However, the potential disadvantages associated with the long-term use of such pharmacological substances may render such a modality problematical.

Studies using primates and other animal models have shown that optical interventions that manipulate the amount of light reaching the eye can induce a shift to myopia. Other studies have shown that optical defocus in young primates can cause the eye to change its growth patterns so that either myopia or hypermetropia (long-sightedness) can be induced by the wearing of negatively-powered or positively-powered spectacle lenses, respectively. For example, when the image is positioned by the use of negative-powered lens to a position posterior to the retina, for example, behind the retina, myopia is induced. This myopia progression is actuated by axial elongation (growth bringing about a “lengthening” of the eye-ball).

Such evidence has prompted the use of bifocal or progressive spectacles or bifocal contact lenses as strategies for retarding the progress of myopia in individuals. However, to date, studies show the efficacy of these strategies to be limited. In the case of spectacle bifocals, compliance of the wearer to always look through the near addition portion for near work cannot be guaranteed. The bifocal contact lenses that have been used to date have been simultaneous vision bifocals. However, such bifocals are known to produce visual problems such as haloes, glare and ghosting, making them undesirable for the wearers.

Additional studies have shown that interrupting myopia-inducing stimuli, for even relatively short periods of time, reduces or even eliminates the myopia-inducing effects of such stimuli. Therefore, a ‘daily-wear’ approach whereby the myope ceases to use the myopia-reduction device for certain periods during the day would not be efficient and may well compromise its efficacy.

Another optical method, used in attempts to retard the progression of myopia in individuals is ‘under-correction’. In under-correction, the wearer is prescribed and provided with a correction (e.g. spectacles, or contact lenses) that is lower in power than the full refractive prescription required for clear vision. For example, a −5 D myope may be given only a −4 D pair of spectacles rendering this myope still −1 D relatively myopic. Therefore, this method implicitly requires the visual image to be blurred or degraded in some way. This detracts from the usefulness of the device as the wearer is constantly reduced in visual performance, (e.g. preventing the wearer from driving due to legal vision requirements). Further, there is evidence to suggest that an under-correction approach may even accelerate myopia progression. A means of abating, retarding, and ultimately reversing, the progression of myopia, would provide enormous benefits to the millions of people who suffer from myopia.

### SUMMARY OF THE INVENTION

The present invention provides a method of abating, retarding or eliminating the progression of myopia or hypermetropia, in an individual by controlling aberrations, thereby manipulating the position of the medium- and high-spatial frequency peaks of a visual image in a predetermined fashion, thereby reducing or eliminating accommodative lag and ultimately altering, reducing or eliminating eye axial elongation.

Further, for the method of the present invention to be maximally effective, as discussed previously, the manipulation is presented to the myope substantially continuously, to cover all open eye situations. Yet further, in another embodiment for optimal control of aberrations, the method of the present invention provides a device that consistently remains relatively coaxial (have substantial centration) with the optics of the eye.

The present invention is also directed to a method by which myopia progression may be retarded (and in many cases, halted or reversed) with the use of a novel optical device having a predetermined aberration controlled design that abates, retards or eliminates eye growth.

Still further, according to the present invention, the progression of myopia is modified by precisely controlling of the optical aberrations of the corrective device, or the combined optical aberrations of the eye and corrective device, such that the medium-spatial frequency peaks are positioned either close to, or more posterior to (i.e. “behind”), the high-spatial frequency peaks. This arrangement eliminates accommodative lag, which is a stimulus for eye axial elongation leading to myopia. Since the device does not introduce significant defocusing (as are, for example, introduced by under-correction methods, or bifocal or progressive optical devices) the devices of the present invention provide the wearer with a good quality visual image. Thus, the invention offers the benefits of retarding progression of refractive error while substantially simultaneously maintaining a clear, useful visual image for the wearer. For purposes of clarity, according to the present invention, the term “behind” orientationally reflects the concept that a point is located at a greater distance from the cornea (and towards the retina) than is another comparative point.

The aberration control aspect of the current invention may be implemented via any suitable optical devices, including, for example, spectacles, contact lenses, orthokeratology (a specialized contact lens technique which aims to alter the refractive state of an eye by remodeling the cornea and epithelium through the short-term wearing of contact lenses of specific designs), corneal implants (e.g. on-lays or in-lays), anterior chamber lenses, and intraocular lenses (IOL), alone or in combination. Preferably, the devices of the present invention are implemented in an optical modality that can remain substantially centered to the axis of the eye such as anterior chamber lenses, IOL, refractive surgery (e.g. epikeratophakia, thermoplasty, LASIK, PRK, LASEK), corneal implants and contact lenses and orthokeratology. In this way, the precise control of aberration leading to the precise, predetermined manipulation of the positions of the spatial frequency peaks could be predictably maintained irrespective of eye movement.

In one embodiment, the present invention is implemented in a contact lens (soft or rigid or scleral haptic type) wear modality, or contact lens used in an orthokeratology modality or corneal on-lay modality, since changes in power and aberration profiles (required as the wearer's amount of myopia changes) can be readily made.

In the case of the contact lens or orthokeratology modalities, a new lens can be prescribed and dispensed readily. For the on-lay, the epithelium is scraped away, the existing on-lay removed and a new on-lay affixed in place with the epithelium allowed to re-grow over the device.

The present invention is particularly well-suited for use in an extended wear or continuous wear contact lens modality or contact lens through an orthokeratology modality, thus providing a substantially continuous stimulus for myopia retardation. Typically, extended wear or continuous wear contact lenses, which may be, for example, soft or rigid gas permeable (RGP) lenses, have sufficient oxygen permeability and other properties to permit the lens to be left in the eye during sleep, while still receiving sufficient oxygen from the tarsal conjunctiva to maintain ocular health, despite atmospheric oxygen not being available due to the closed eye-lid.

In orthokeratology, the contact lens (which may also be of the high oxygen permeability kind suitable for extended or continuous or overnight wear) may be worn for a short period (e.g. during sleeping hours) to remodel the epithelium and cornea after which the contact lens may be removed leaving the patient in the desired refractive and aberration state, according to the present invention, without contact lens wear for the period of effectiveness of the orthokeratology.

The present invention can be realized in a number of ways to retard or eliminate myopia such that an ocular device designed with a prescribed amount of suitable aberrations is provided, or a direct and predetermined refractive change is effected such that, in combination with ocular aberrations, the medium spatial frequency peak is located “behind” the high spatial frequency peak. This arrangement affords a continuously clear vision for the wearer while simultaneously promoting retardation in the progression of myopia.

### BRIEF DESCRIPTION OF THE DRAWINGS

FIG. 1 is a plot of a modulation transfer function (MTF) for an optical system.

FIG. 2 is a plot of a through-focus modulation transfer function (MTF) graph for a non-myopic eye.

FIG. 3 is a plot of a through-focus modulation transfer function (MTF) graph for a myopic eye.

FIGS. 4a-4d are diagrams illustrating the effect of accommodative lag on axial elongation with particular relative positioning of spatial frequency peaks.

FIG. 5 is a plot of accommodative gradient versus third-order spherical aberration in a group of subjects demonstrating the link between aberrations and accommodative lag.

FIGS. 6a-6e are diagrams illustrating the through-focus modulation transfer function (MTF) graphs for uncorrected and corrected eyes.

FIGS. 7a-7b are graphs illustrating the optical effect achieved by modifying a soft contact lens using a polynomial series to describe and generate an anterior surface.

FIGS. 8a-8b illustrate the optical effect achieved by combining conic sections and polynomials.

FIGS. 9a-9b illustrate the ability to incorporate any required refractive prescription into the present invention to correct refractive error in an eye.

FIGS. 10a-10g illustrate the ability of the present invention to correct wave-front aberrations while simultaneously controlling the relative position of the spatial peaks.

FIGS. 11a-11b illustrate the relative positioning of spatial peaks in hypermetropes and the positioning shift afforded by aberrations introduced by contact lenses with spherical front and back surface designs.

FIG. 11c illustrates a prescription, thickness, and surface profile for a contact lens design according to one embodiment of the present invention.

### DETAILED DESCRIPTION OF THE INVENTION

There is evidence that the optical stimulus that triggers the progression of myopia is not strictly refractive in the conventional manner (i.e. spherical and astigmatic defocus) as are prescribed by eye-care practitioners such as ophthalmologists, optometrists and opticians, using vision devices such as spectacles, contact lenses, anterior chamber lenses or intra-ocular lenses (IOL). It has been shown that myopes have higher amounts of higher order optical aberrations (e.g. spherical aberration, i.e. not simply defocus or astigmatism), and that myopia is also associated with certain types of optical aberration such as coma. Studies have shown that myopes cannot accommodate as precisely (called “accommodative lag”, as defined later) or as easily as non-myopes. Accommodation is also known to be driven primarily by the medium-spatial frequencies of around 5 cycles per degree (cpd).

We have now shown experimentally that myopes and non-myopes show marked differences in their contrast sensitivity response as a result of blur. Such differences in contrast sensitivity response can be explained by the differences in modulation transfer function (MTF) which may be influenced by differences in aberrations between myopes and non-myopes.

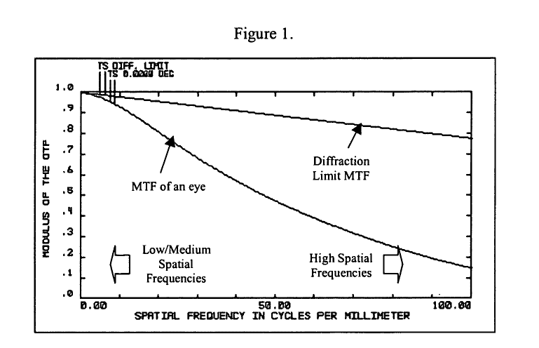

It is known that with certain types and combinations of aberrations of an optical system, including the eye, different amounts of high—(above around 15 cpd), medium—(of around 5 cpd) and low—(below around 5 cpd) spatial frequencies are transmitted with different levels of fidelity (or “modulus”, quantified as an index from 0 to 1 with 0 being a total loss of signal, and 1 representing no loss of signal or fidelity) to different positions along the optical visual axis. As shown in FIG. 1, the performance of an optical system, including the human eye, can be portrayed by plotting its MTF. Such an MTF graph is shown illustrating the performance of one eye example. The curve on the graph shows the relative ability of the eye to transmit information of various spatial frequencies: high spatial frequencies, towards the right of the graph, (representing the very fine visual details) and medium and lower spatial frequencies, towards the left of the graph, (the coarser visual details). These spatial frequencies are plotted along the horizontal axis. For the human eye, 100 cycles per millimeter corresponds approximately to 30 cycles per degree, which is nominally equivalent to 20/20 visual acuity. The ability of the eye to reproduce/transmit each frequency to the retina is represented by the MTF curve. The better the performance of the eye, the higher the MTF curve tends to be. The MTF curve of the ‘perfect’ eye will be identical to the “diffraction limit” curve.

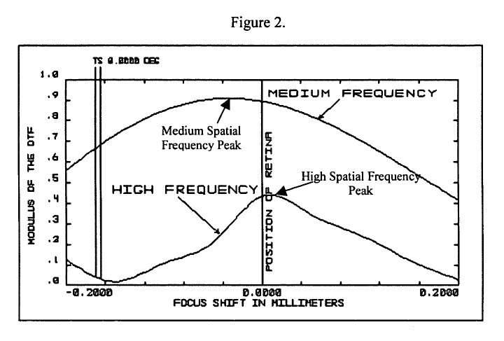

The consequence of this differential transmittance of the different spatial frequencies is that the MTF peaks (maxima) of different spatial frequencies differ in their axial positions. Such differences can be illustrated by a ‘through-focus’ MTF graph. As shown in FIG. 2, the amount that each spatial frequency is transmitted also depends on the axial position (distance along the eye towards the retina). Such a graph is shown for a non-myopic eye. For a given spatial frequency, as axial position changes (along the horizontal axis), the modulus of transfer for that spatial frequency also changes. Typically, a peak modulus (maxima) can be identified for each spatial frequency. Such a spatial frequency peak may be located on the image plane (the retina), in front of the image plane (more anteriorly or closer towards the cornea), or behind the image plane (more posteriorly or further from the cornea). The peaks of different spatial frequencies are not always located in the same axial position. Therefore, our experimentation supports the postulate that for non-myopes, the axial positions of their high and medium spatial frequency peaks are typically positioned such that the medium spatial frequency peaks are located more posteriorly relative to (e.g. “behind”) the high spatial frequency peaks. In addition, in cases where the medium spatial frequency peaks are located more anteriorly than (e.g. “in front of”) the higher spatial frequency peaks, the medium and high spatial frequency peaks are close together; i.e. a distance apart from one another of typically less than the equivalent refractive power difference of around 0.25 D (FIG. 2).

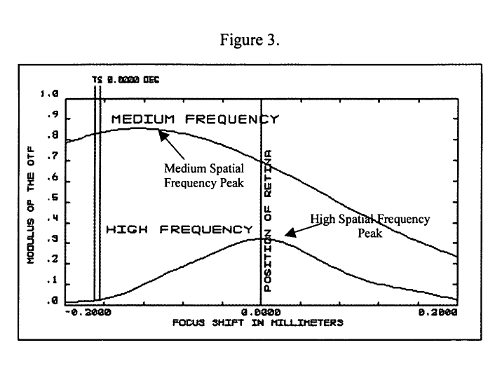

We further postulate that for myopes and eyes with myopic tendencies (i.e. not yet myopic, but would develop into myopia), given their different aberrations, the axial position of the high spatial frequency peak is located well posteriorly relative to the medium spatial frequency peak (more than about 0.25 D apart). As shown in FIG. 3, a through-focus MTF graph is plotted for an eye with myopic tendencies (i.e. already myopic, or may become myopic). In this example, the 5 cpd medium frequency peak (approximately 0.85 modulus) is located approximately 120 μm anterior (i.e. towards the front of the eye, or the cornea) to the 25 cpd high frequency peak (approximately 0.35 modulus). This difference in axial positions equates to a refractive power difference, in this example, of approximately 0.35 D.

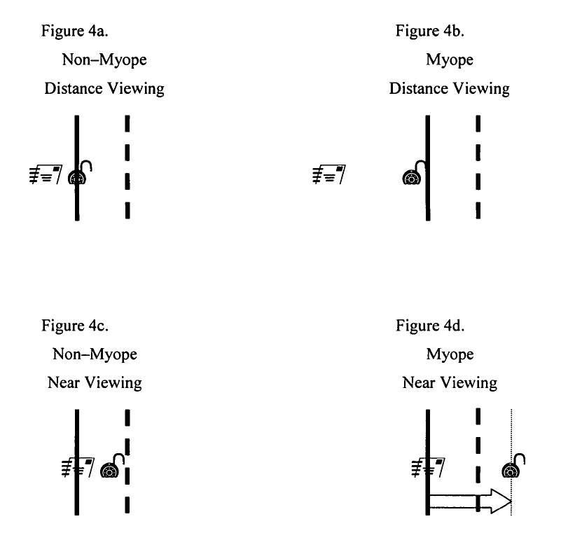

We yet further postulate that axial length elongation, as part of eye growth, for example, during induction of myopia, is driven by the position of the high spatial frequency peak. During near work such as reading, focusing of the eye (a process called “accommodation”) is effected by a change in the shape of the crystalline lens, thus increasing the focusing power of the eye. Since accommodation is driven by the medium spatial frequencies, that focus is set such that the medium spatial frequency peak will be positioned on the retina. However, since the high spatial frequency peak for the myope is consequently more posteriorly positioned, this provides a stimulus for the eye to grow, resulting in axial elongation and the induction or progression of myopia. The difference in accommodative focus due to the medium spatial frequency visual contents driving accommodation and that required for viewing high frequency visual contents is the accommodative lag. FIGS. 4a-4d illustrate the effect of accommodative lag on axial elongation with particular relative positioning of spatial frequency peaks. In these diagrams, the high spatial frequency peaks are represented by symbol [symbol] , medium spatial frequency peaks by symbol [symbol] , the retina by a solid vertical line, and acceptable tolerance in differences in positions between spatial frequency peaks before stimulus to growth is induced, is represented by a broken vertical line behind the retina. For orientation, in these figures, the front of the eye (e.g. the cornea) is towards the left, and light enters and travels through the eye from the left (front) to the right (back).

For distance viewing, the eye is focused with the high spatial frequency peak near or on the retina. For the myope without refractive correction, both the high and medium spatial frequency peaks are positioned in front of the retina during distance viewing as is typical of this refractive condition. This is illustrated (FIG. 4a) for a non-myope or emmetrope (a person who is neither longsighted nor shortsighted), and an eye with myopic tendencies (FIG. 4b).

During near work such as reading, the eye is refocused to the nearer visual object by increasing its focusing power. Since accommodation is driven by the medium-spatial frequencies, near focus is set such that the medium-spatial frequency peak will be positioned on the retina. In this situation, the difference in focal positions between the medium- and high-spatial frequency visual contents represents the accommodative lag of the eye. This is shown for the non-myope (FIG. 4c), and myope (FIG. 4d).

However, since the high spatial frequency peak, which drives axial growth, is now more posteriorly positioned for the myope, and beyond the tolerance for triggering growth, a stimulus for the eye to grow (in the direction of the arrow) is evoked in order to attempt to place the retina onto the high spatial frequency peak. This results in axial elongation and the induction or progression of myopia.

This foregoing explanation is consistent with the study results discussed above concerning myopic progression. For example, under-correction of a myope may not retard progression, and indeed may induce further progression, just as the use of spectacle lenses may actually increase aberrations that further separate the positions of the medium and high spatial frequency peaks. Similarly, the use of progressive or bifocal spectacles would not alter myopia progression if the optics of those devices is such that the relative positions of the medium and high spatial frequency peaks remain unaltered. Indeed, in some cases, these devices may further increase the separation of the medium and high spatial frequency peaks, thereby further driving the progression of myopia. From the aforementioned, it is the contention of this invention that one cause of myopia induction and progression is a result of the optical aberrations of myopes, and those with myopic tendencies, causing the differential axial positioning of the medium and high spatial frequency peaks, which during near focusing, introduces accommodative lag (due to accommodation being driven by medium spatial frequencies), leading to the positioning of the higher-spatial frequency peaks behind the retina, ultimately triggering axial elongation and myopia progression.

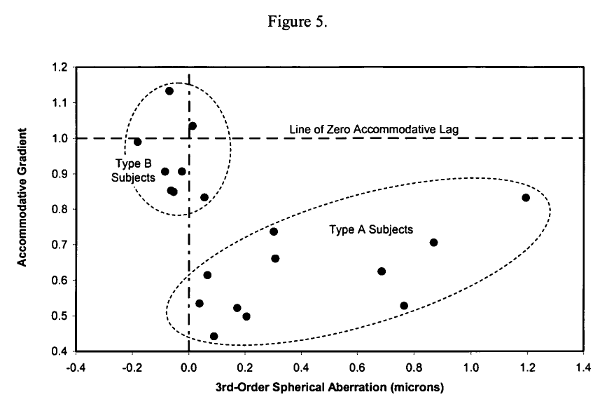

In our studies, we have demonstrated this relationship in a group of subjects. In this study, patients were required to focus (accommodate) through a series of increasingly powerful negative powered lenses while simultaneously, the actual amounts of accommodation exerted by the patients' eyes were measured. If no accommodative lag exists, then a plot of the amount of accommodation against the power of the negative lenses, called the “accommodative gradient” would return a line with a slope of 1. That is, for every diopter of negative optical power induced in front of the eye, the eye would accommodate 1 diopter in response. (In practice, there is some measurement error involved so the actual measured slope may be slightly different from 1.) Should accommodative lag be present, the accommodative gradient would be less than 1. That is, the eye is not accommodating the full amount demanded by the negative lens. In the same study, for each subject, we also measured the amount of third-order spherical aberrations, a type of optical aberration of the eye. The result of our study is shown in FIG. 5. In this graph, the accommodative gradient is plotted against the subjects' third-order spherical aberration. It can be clearly seen that for those subjects who have spherical aberration greater than around 0 μm there is an appreciable amount of accommodative lag present. In contrast, subjects with less than around 0 μm spherical aberration showed effectively no accommodative lag.

Third-order spherical aberration is one way by which the relative axial positioning of high and medium spatial frequency peaks can be altered, and our study showed that it is correlated to accommodative lag which in turn, as explained above, can lead to the development and progression of myopia, Therefore, the basis for the present invention is formulated. By manipulating or controlling the aberrations of the eye, or the combined eye and corrective optical system, the relative axial positions of the high and medium spatial frequency peaks can be manipulated or controlled so as to reduced or eliminate accommodative lag, thereby eliminating the stimulus for axial growth, and in turn reducing or eliminating the onset, development or progress of myopia.

The present invention provides a method of retarding or eliminating the progression of myopia in an individual by controlling aberrations and stimuli presented to an eye, thereby manipulating the positioning of the medium and high spatial frequency peaks of a visual image, thereby reducing or eliminating accommodative lag and ultimately reducing or eliminating eye axial elongation.

Further, for this method to be maximally effective, as discussed previously, the predetermined correction and aberration designs are preferably presented to the myope substantially continuously, to cover all open eye situations. Yet further, for optimal control of aberrations, the method must provide a device that consistently remains substantially coaxial (having substantial centration) with the optics of the eye. The present invention also provides a method by which myopia progression may be abated, retarded, and in many cases halted or reversed, with the use of novel optical devices and systems that retard or eliminate eye growth.

The methods and apparatuses of the present invention modify the progression of myopia by precisely controlling, in a predetermined fashion, the optical aberrations of the corrective device, or the combined optical aberrations of the eye and corrective device, such that the medium-spatial frequency peaks are positioned more posteriorly than the high-spatial frequency peaks. This arrangement eliminates accommodative lag, thereby removing the stimulus for eye axial elongation and myopia progression.

Since the device does not introduce any defocusing effects, as are introduced by under-correction methods, or bifocal or progressive optical devices, this device provides the wearer substantially simultaneously with a good quality visual image. Thus, the invention offers the benefits of retarding progression of refractive error while simultaneously maintaining a substantially continuous, clear, useful visual image for the wearer.

While the aberration control aspect of the current invention may be implemented in any suitable optical devices including spectacles, contact lenses, orthokeratology (a specialized contact lens technique which seeks to alter the refractive state of the eye by remodeling the cornea and epithelium by the short-term wearing of contact lenses of specific designs), corneal implants (e.g. on-lays or in-lays), anterior chamber lenses, intraocular lenses (IOL), etc., as well as by surgical refractive procedures (e.g. epikeratophakia, thermoplasty, LASIK, PRK, LASEK, etc.), the aberration control is preferably implemented in an optical modality that can remain relatively centered to the axis of the eye such as an anterior chamber lenses, IOL, corneal implants, contact lenses, orthokeratology or refractive surgery. In this way, the precise control of aberration leading to the precise, predetermined manipulation of the positions of the spatial frequency peaks can be maintained irrespective of eye movement.

Further, the present invention is more preferably implemented in a contact lens (soft or rigid or scleral haptic type) wearing modality or contact lens used in an orthokeratology modality or a corneal on-lay modality since changes in power and aberration profiles (required as the wearer's amount of myopia changes) can be readily made.

In the case of contact lenses including contact lenses used in the orthokeratology modality, a new lens can be prescribed and dispensed readily. For the on-lay, the epithelium is scraped away, the existing on-lay removed and a new on-lay affixed in place and the epithelium is allowed to re-grow over the device.

Even further, the present invention is most preferably implemented in an extended wear or continuous wear contact lens, or orthokeratology modalities, thus providing a substantially continuous stimulus for myopia retardation.

Typically, extended wear or continuous wear contact lenses, which may be soft or rigid gas permeable (RGP), have sufficient oxygen permeability and other properties to permit the lens to be left in the eye during sleep and still receive sufficient oxygen from the tarsal conjunctiva to maintain ocular health despite atmospheric oxygen not being available due to the closed eye-lid.

For orthokeratology, the contact lens (which may also be of the high oxygen permeability kind suitable for extended or overnight wear) is worn for a short period (e.g. during sleeping hours) to remodel the epithelium and cornea after which the contact lens is removed leaving the patient in the desired refractive and aberration state according to the present invention without contact lens wear for the period of effectiveness of the orthokeratology. The contact lens design for use with the orthokeratology modality has a dual role. The contact lens is designed such that when worn during the ‘treatment’ or remodeling period, the combined eye and contact lens aberrations are manipulated according to the present invention. Further, the lens back or posterior surface profile, together with the lens rigidity and thickness profile, all of which controls the remodeling of the epithelium and cornea, can be manipulated so that upon lens removal (after the lens wearing ‘treatment’ period of orthokeratology), the remodeled cornea and epithelial profile is such that the residual ocular aberrations is controlled according to the present invention.

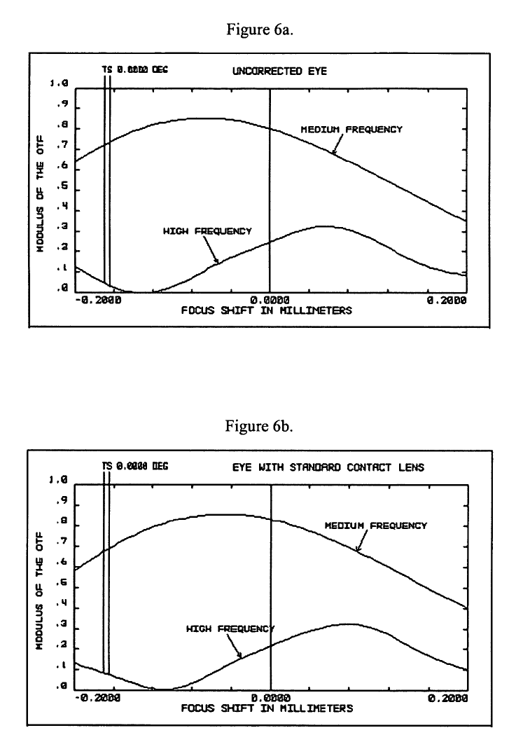

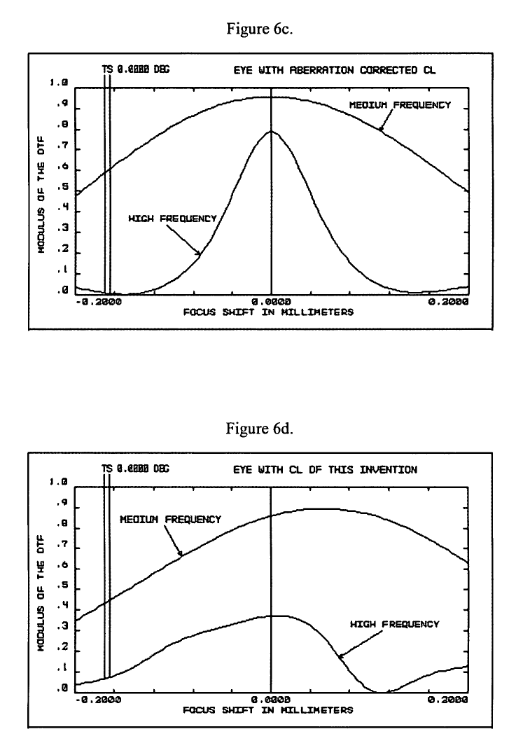

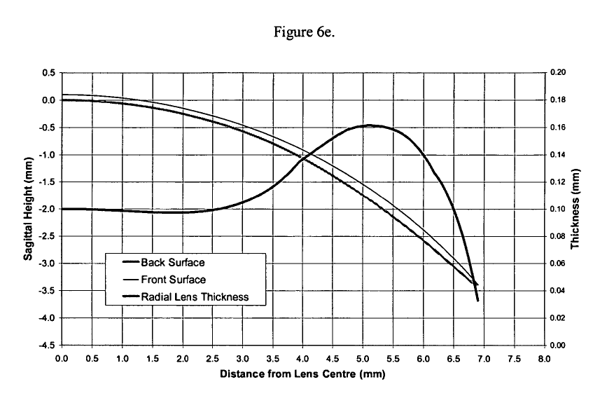

The prescription and “through focus MTF” graph, which shows the axial positions of the medium and high spatial frequency peaks, of one example of this embodiment which employs a conic section profile for its optical surfaces is shown in FIG. 6.

It should be noted that the design of such a contact lens differs substantially from those designed for the optimization of vision by the correction of aberrations. When a lens is designed to substantially reduce or eliminate the aberrations of the eye, including what are called the “higher order aberrations”, such as to provide above-normal visual performance (sometimes referred to as “super-vision”), the axial positions of the medium- and high-spatial frequency peaks are very close together. By contrast, according to the present invention, for the retardation or elimination of myopia progression, the medium-spatial frequency peaks are preferably located “behind” (more posteriorly to) the high-spatial frequency peaks.

The present invention can be realized in a number of ways, such that an ocular device designed with a prescribed and predetermined amount of suitable aberrations is provided, or a direct and predetermined refractive change is effected, such that the medium-spatial frequency peak is located behind the high-spatial frequency peak. This arrangement affords a continuously clear vision for the wearer while promoting retardation in the progression of myopia. The prescription and “through focus MTF” graph, which shows the axial positions of the medium and high spatial frequency peaks, of one example of this embodiment which employs a conic section profile for its optical surfaces is shown in FIGS. 6a-6d. The through-focus MTF for a high (25 cpd) and a medium (5 cpd) spatial frequency is shown for an eye with myopic tendencies. Such an eye has its high spatial frequency peaks significantly more posteriorly positioned than the medium spatial frequency peaks (See FIG. 6a).

When a standard contact lens using conventional spherical front and back surfaces is used to correct this eye, the outcome is shown in the through-focus MTF graph in FIG. 6b. Note that there is no substantial change in the relative distances and axial positions of the two spatial frequency peaks on the application of such a standard design contact lens.

A more recent approach, to achieve above-normal vision (or super-vision) is to reduce or eliminate the aberrations of the eye and contact lens by producing aberration corrected designs. The outcome through-focus MTF graph is shown in FIG. 6c. In this case, the axial positions of the two spatial frequency peaks have been ‘collapsed’ to one location. While this design may provide excellent vision, it would be insufficient in retarding, eliminating or reversing the progression of myopia in the wearer.

Thus the key aspect to the present invention is not ascribed explicitly to the spherical aberrations involved in any optical design, but the relative positioning of the high and medium spatial frequency peaks to be achieved.

Thus, according to the present invention, by designing the appropriate amount and type of aberrations into a contact lens, for example, by employing conic-section aspheric surfaces for both the anterior and posterior contact lens surfaces, the through-focus MTF shown in FIG. 6d can be achieved. Note that whereas before contact lens correction the eye had a high spatial frequency peak posterior to the medium spatial frequency peak, in this novel design, the medium spatial frequency peak is now more posteriorly located relative to the high spatial frequency peak. This arrangement will promote the retardation and elimination, and potentially reverse progression, of myopia in the wearer as the stimulus for axial elongation during near work has been totally removed.

In this example, the eye is emmetropic and hence would require simply a Plano (zero refractive power, or 0 D) correction. However, the effect can still be achieved as illustrated in the foregoing through the appropriate choice and application of aspheric surface designs. The prescription, thickness profile and surface profile for the contact lens design of this particular example are shown in FIG. 6e. The anterior surface central radius (also called the “front optic zone radius” or FOZR) is 8.196 mm with an asphericity of k=−0.51, a central thickness of 100 μm and a posterior surface central radius (also called the “back optic zone radius” or BOZR) of 8.30 mm with an asphericity of k=0.45. The optic zone diameter (OZD) is 8.00 mm. The refractive index of the lens is assumed to be that of hydrated hydroxyethylmethyacrylate (HEMA), a commonly used soft contact lens material well known to those skilled in the ocular science field.

It will become apparent to readers of the description of the foregoing embodiments that the manipulation of the relative positions of the medium and high spatial frequency peaks using a controlled amount of aberration may be achieved in several ways. For example, instead of the use of conic sections to define the profiles of the optical surfaces, other surface descriptors may be used including polynomials, combinations of conic sections and polynomials, splines, Bezier functions, Fourier series synthesis, Zernike polynomial as sagittal height descriptors, or a more general point-by-point surface description via a look-up-table or similar approaches. Further, the design of optical devices of the present invention is not limited to the controlling of optical surface profiles. For example, gradient refractive index (GRIN) materials may be used to manipulate the relative positions of the medium- and high-spatial frequency peaks, as may Fresnel-type optics, holographic or diffractive optics be used, either individually or in combinations with each other or with the surface profile design approaches.

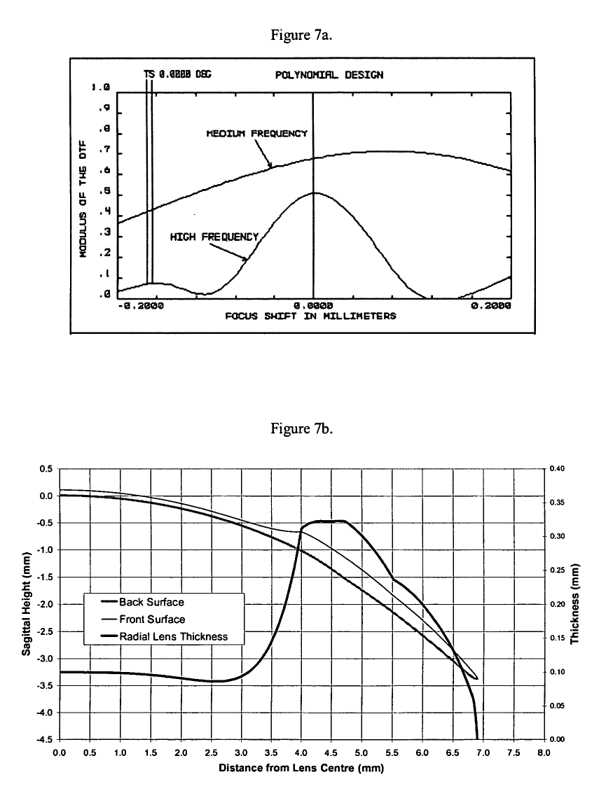

In FIG. 7a, the optical effect is achieved by modifying the profile of a soft contact lens by employing a polynomial series to describe and generate the anterior surface. This results in the appropriate relative positioning of the medium spatial frequency peak behind the high spatial frequency peak by approximately 80 μm (equivalent to around 0.25 D). The prescription, thickness profile and surface profile for the contact lens design of this particular example are shown in FIG. 7b. The FOZR in this case is described by a basic sphere of radius 8.312 mm with additional sagittal height departures from this basic sphere described by a polynomial equation of the form s(x)=a.x²+b.x⁴+c.x⁶+d.x⁸ where x is the distance from the contact lens axis in millimeters, a=0.000160, b=0.000052, c=−0.000014, and d=−0.000005. The OZD is 8.00 mm. The central thickness is 100 μm and the back surface is a sphere with a BOZR of 8.30 mm. The refractive index of the lens is assumed to be that of HEMA.

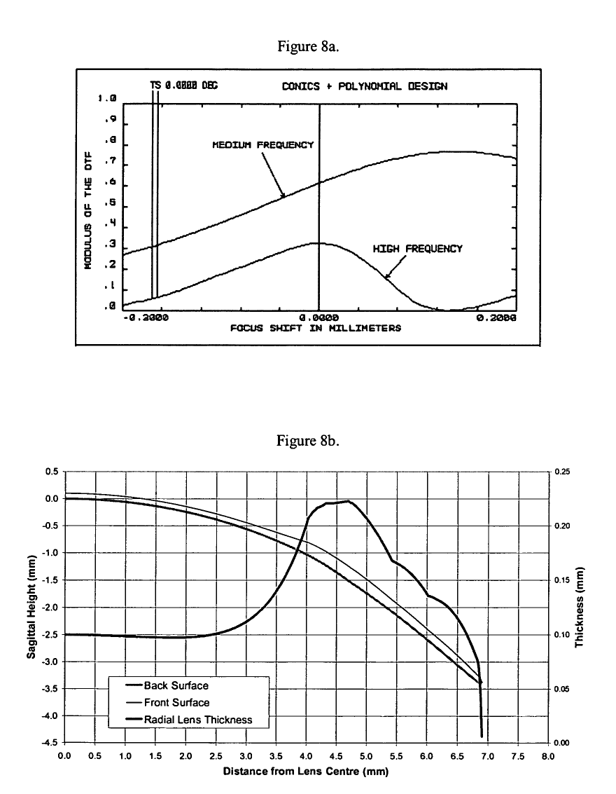

By combining conic sections and polynomials, the greater degrees of freedom available in defining the surface profile can provide greater optical effect in terms of the appropriate relative positioning of the spatial frequency peaks. In FIG. 8a, a design for a contact lens of the present invention employs a combination of a conic section and polynomials to realize a separation of the spatial frequency peaks by approximately 150 μm (equivalent to around 0.4 D). The prescription, thickness profile and surface profile for the contact lens design of this particular example are shown in FIG. 8b. The FOZR in this case is described by a basic conic section of central radius 8.197 mm and asphericity (k factor) of −0.95 with additional sagittal height departures from this basic sphere described by a polynomial equation of the form s(x)=a.x²+b.x⁶+c.x⁸ where x is the distance from the contact lens axis in millimeters, a=0.000128, b=−0.000004, and c=−0.000001. The OZD is 8.00 mm. The central thickness is 100 μm and the back surface is a sphere with a BOZR of 8.30 mm. The refractive index of the lens is assumed to be that of HEMA.

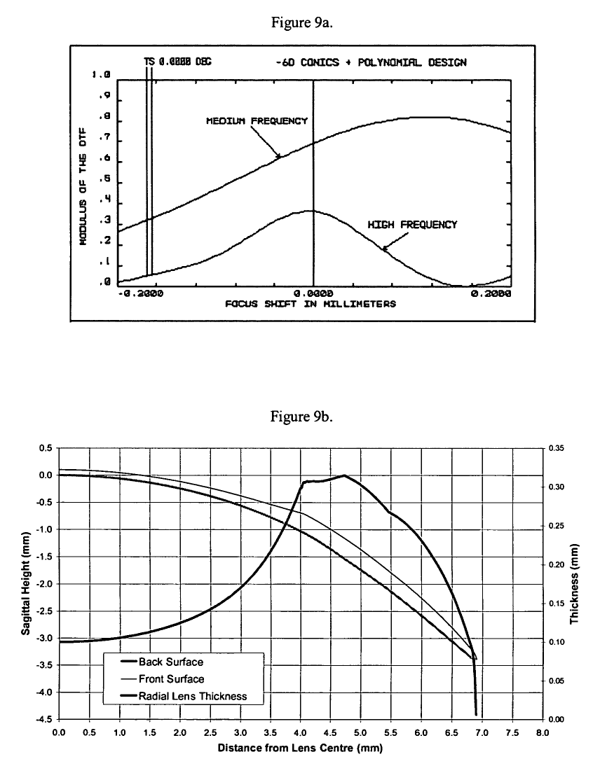

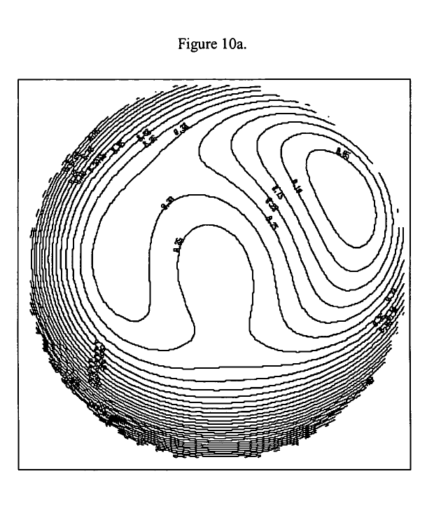

The present invention further contemplates that a device of the current invention may be designed to incorporate any refractive prescription required to correct the existing refractive error of the eye. For example, a −6 D prescription may be introduced to the device, then the suitable amount of aberrations added to reposition the medium and high spatial frequency peaks appropriately, thereby providing continued good corrected vision for the −6 D myopic wearer while retarding the progression of his/her myopia. In FIG. 9a, a design for a soft contact lens of the present invention, incorporating a refractive correction for a −6 D myope, employs a combination of conic section and polynomials to realize a separation of the spatial frequency peaks by approximately 120 μm (equivalent to around 0.32 D). The prescription, thickness profile and surface profile for the contact lens design of this particular example are shown in FIG. 9b. The FOZR in this case is described by a basic conic section of central radius 9.279 mm and asphericity (k factor) of −0.95 with additional sagittal height departures from this basic sphere described by a polynomial equation of the form s(x)=a.x²+b.x⁴+c.x⁶+d.x⁸ where x is the distance from the contact lens axis in millimeters, a=0.000186, b=0.000005, c=−0.000003 and d=−0.000001. The OZD is 8.00 mm. The central thickness is 100 μm and the back surface is a sphere with a BOZR of 8.30 mm. The refractive index of the lens is assumed to be that of HEMA.

It should now be clear, given the foregoing description, that it is also possible to correct astigmatism in an eye while retarding the progression of myopia in a wearer.

Designs for regular astigmatism can be treated simply as a design for two spherical refractive power corrections of different power and along two perpendicular axes on the same eye and optical corrective device. For example, to correct a wearer with a prescription (written in the “minus-cylinder form” as would be understood by vision care practitioners such as opticians, optometrists and ophthalmologists) of −6 D/−2 D×180, the design approach would merely be to treat the vertical (90 degree axis) and horizontal (180 degree axis) separately. A −6 D correction is designed for the vertical axis along the same principle as described previously. A −8 D correction is designed for the horizontal axis also along similar principle as previous. As understood by vision care practitioners, corrections for astigmatism would require the devices to maintain their axis orientation with respect to the eye. A number of design configurations and features are well known to the practitioners for achieving such orientational alignment. For example, in the case of contact lenses, prism ballasts, slab-off designs and truncations may be used.

Correction of irregular astigmatism may be regarded as a special case of correction of wave-front aberrations and is described below.

One advanced approach in vision correction provides for the correction of the wave-front aberrations (typically including higher-order aberrations) of the eye. A lens design of the present invention may incorporate partial wave-front aberration correction while simultaneously controlling the position of the medium spatial frequency peaks to be more posterior than the higher spatial frequency peaks. This approach can provide further improved vision while maintaining the stimulus that is required to retard the progression of myopia.

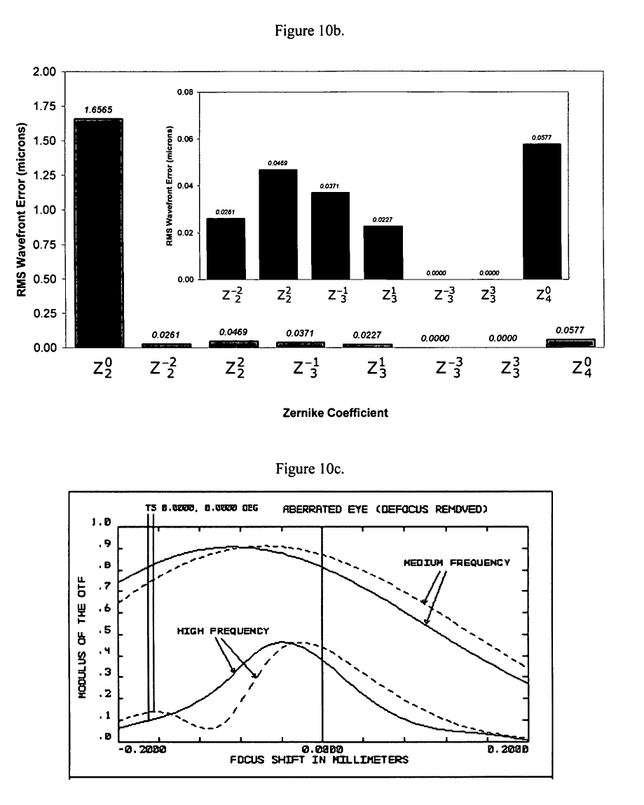

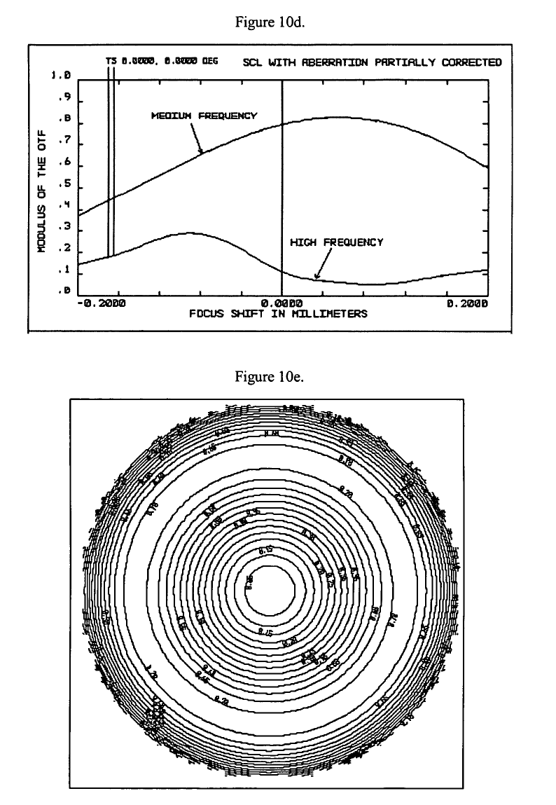

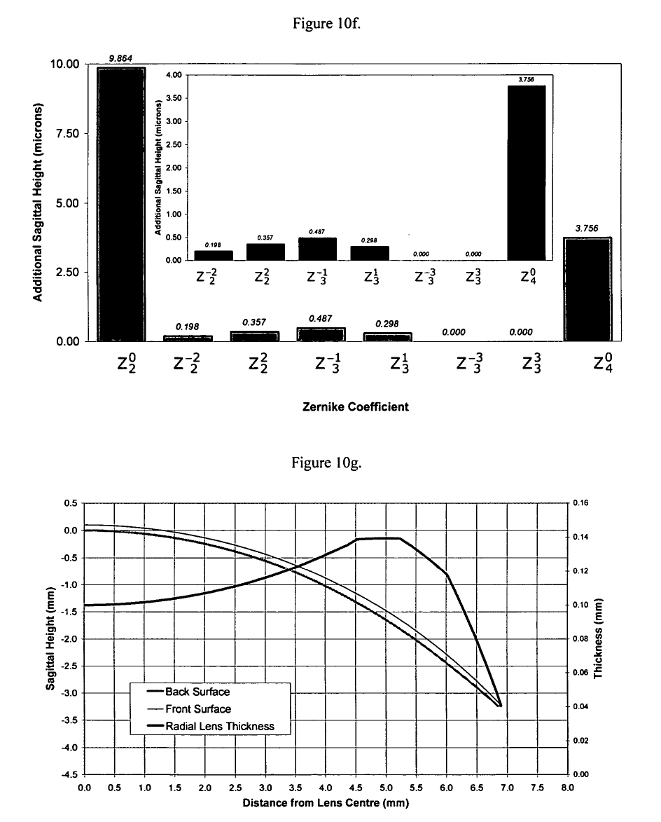

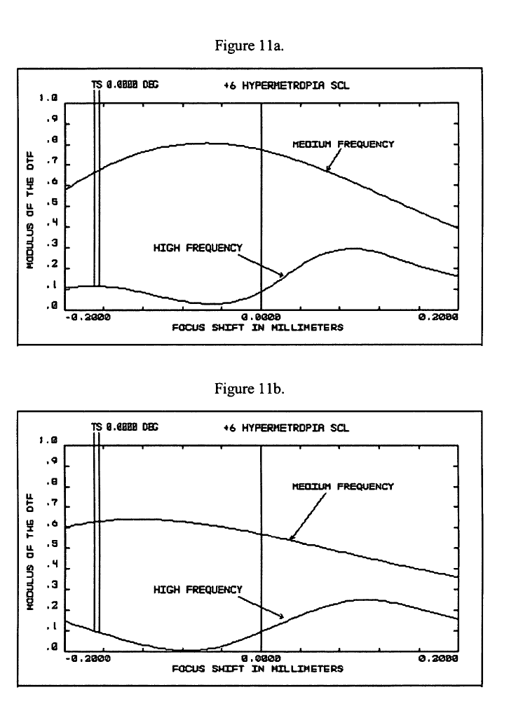

The aberrations of an individual may be measured using a range of ocular wave-front sensors (e.g. Hartmann-Shack devices). An example of an individual's wave-front aberration is shown in FIG. 10a. The defocus effect has been removed in this wave-front map in order to reveal the higher order aberrations more clearly. For quantitative analyses, vision scientists and optical engineers may describe wave-front aberrations as a Zernike polynomial series. An additional advantage of this method of describing aberrations is that the Zernike polynomial terms relate to aberration-types familiar to the optical engineer or vision scientist. For example, coefficient Z₂⁰ is indicative of defocus in the optics of the eye and Z₃¹ is indicative of the presence of coma (a type of aberration) in the optics of the eye. The RMS wave-front error associated with each of the Zernike polynomial terms up to Z₄⁰ is shown in FIG. 10b. It can be seen that for this particular individual, significant amounts of defocus (in this case, myopia) is present. The inset in FIG. 10b shows the higher order Zernike terms with defocus removed in order to show them with greater precision. From the inset, it can also be seen that this individual has discernible amounts of astigmatism (Z₂⁻² and Z₂²), coma (Z₃⁻¹ and Z₃¹) and spherical aberrations (Z₄⁰). The through-focus MTF graph for this individual's eye with defocus removed is shown in FIG. 10c. Since this eye has an amount of astigmatism, two through-focus MTF curves are shown, one for each of the line foci associated with the astigmatism. However, it can be seen that for both line foci, the medium spatial frequency peaks are located more anteriorly to the higher spatial frequency peaks as is typical of eyes with myopic growth tendencies.

Total correction of the wave-front aberrations of this eye would result in the co-location of the medium and higher spatial frequency peaks similar to that seen in FIG. 6c. This is unsuitable for the retardation and reversal of myopia progression.

A soft contact lens designed according to the principles of the present invention can reposition the medium spatial frequency peaks more posteriorly to the higher spatial frequency peaks while partially correcting the higher-order aberrations of the eye. This arrangement would promote the retardation and potential reversal of myopia progression while providing some of the additional benefits of aberration correction. The through-focus MTF graph of one such arrangement is shown in FIG. 10d. By the judicious, partial correction of aberrations, the medium spatial frequency peak is now positioned approximately 150 μm (equivalent to around 0.38 D) more posteriorly as compared to the higher spatial frequency peak. The resultant wave-front error map of the soft contact lens and eye combined (FIG. 10e) shows only concentric rings indicating that coma and astigmatism have been effectively eliminated.

Since the wave-front aberration of the eye in this example is rotationally asymmetrical, the lens design example of this invention is also rotationally asymmetrical (in this case, in order to correct astigmatism and coma). The description of such a lens design may also be expressed as a series of Zernike polynomial coefficients. This is shown in FIG. 10f. Here, the Zernike polynomial series represents additional sagittal heights, i.e. thickness to be added to the spherical front surface of a soft contact lens (FIG. 10g) with FOZR of 8.70 mm. The OZD is 8.00 mm. The back surface of the soft contact lens has a BOZR of 8.35 mm, with a central thickness of 100 μm. The refractive index of the lens is assumed to be that of HEMA.

Due to the rotation asymmetry of this design, the device would need to maintain the correct axis orientation with respect to the eye in the same way as the device for correcting astigmatism (described above). The same design configurations and features as described for correcting astigmatism may be used.

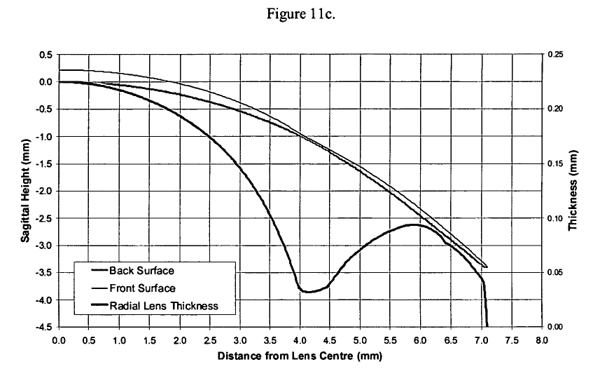

It may be desirable for hypermetropes to induce eye growth and axial lengthening in order to reduce the amount of hypermetropia, or to return fully to emmetropia. Conventional contact lenses with spherical front and back surface designs, due to their lens form, already provide some amount of aberration which results in the high spatial frequency peaks being positioned more posteriorly than the medium spatial frequency peaks (FIG. 11a). Hence, some stimulus already exists for inducing eye axial growth. However, the approach of the present invention indicates that significantly accelerated eye growth, stimulating significantly more rapid return towards emmetropia, can be realized by incorporating additional aberrations to position the high-spatial frequency peaks even further behind the medium-spatial frequency peaks.

For example, a +6 D prescription for a hypermetrope may be incorporated in a device, with the suitable amount of aberrations then added to reposition the medium and high spatial frequency peaks appropriately, thereby providing continued good corrected vision for the +6 D hypermetropic wearer while reducing or eliminating hypermetropia. In FIG. 11b, a design for a contact lens of the present invention incorporating a refractive correction for a +6 D hypermetrope employs a combination of conic section and polynomials to realize an even greater separation of the spatial frequency peaks than achievable with standard, conventional spherical surface contact lenses.

In this configuration (FIG. 11b), the spatial frequency peaks are separated by over 240 μm (equivalent to around 0.65 D) in contrast to the conventional design (FIG. 11a), which could only provide a separation of around 150 μm (equivalent to around 0.4 D).

The prescription, thickness profile and surface profile for the contact lens design of this particular example are shown in FIG. 11c. The FOZR in this case is described by a basic conic section of central radius 7.769 mm and asphericity (k factor) of 0.09 with additional sagittal height departures from this basic sphere described by a polynomial equation of the form s(x)=a.x²+b.x⁴+c.x⁶+d.x⁸ where x is the distance from the contact lens axis in millimeters, a=−0.000116, b=−0.000003, c=0.000002 and d=0.0000008. The central thickness is 225 μm and the back surface is a sphere with a BOZR of 8.60 mm. The OZD is 8.00 mm. The refractive index of the lens is assumed to be that of HEMA.

The key requirement is that the designs of the present invention will afford useful vision while simultaneously posteriorly repositioning the medium spatial frequency peaks, preferably to a location “behind” the high spatial frequency peaks. The present invention further contemplates that the present methods and apparatuses may be applied to any prescription required to correct the existing refractive error of the eye. For example, a −6 D prescription may be introduced to the device, with the suitable amount of aberrations then added to reposition the medium and high spatial frequency peaks, thereby providing continued good corrected vision for the −6 D myopic wearer while retarding the progression of his/her myopia.

The invention may be realized as mass-produced devices, for example by high volume molding technology, or as custom-designed devices. In the case of mass-produced devices, the aberration may be designed to be suitable for the typical sub-population of myopes. For example, for a mass production −3 D prescription device intended for retarding the progression of −3 D myopes, the aberration design would include compensation for the aberrations of a typical −3 D myope. Useful effects can be achieved by population-average mass-produced designs in many individuals. However, for a given individual, optimal myopia retardation effect is produced by the custom-designed devices. For the custom-designed devices, the actual ocular aberrations of the individual intended wearer may be measured, for example using one of a range of ocular wavefront sensors (e.g. Hartmann-Shack devices). The design then takes into account the actual aberration in addition to the aberrations required to reposition the medium and high spatial frequency peaks.

The present invention further contemplates promoting the return of a hypermetropic eye towards emmetropia. This is realized by the introduction of a suitable amount of aberration into the device so that the high-spatial frequency peaks are positioned substantially “behind”, or posterior of the medium-spatial frequency peaks, thereby promoting axial elongation and, hence, reduction of hypermetropia.

While the preferred embodiments are in the form of soft or RGP contact lenses, it will be immediately obvious to those skilled in the art that this invention may also be implemented in other forms of contact lenses (e.g. haptic or scleral contact lenses and “piggy-back” systems where two or more lenses are worn in tandem), spectacles, anterior chamber lenses, IOLs, artificial corneas (e.g. in-lays, on-lays, keratoprostheses), anterior chamber lenses as well as refractive surgery (e.g. epikeratophakia, thermoplasty, PRK, LASIK, LASEK, etc.). In the case of RGP or haptic/scleral contact lenses being used, the aberration profile will be designed also to take into account the optical influence of the tear-lens (produced by the tear layer between the posterior surface of the RGP and the anterior cornea).

With the potential introduction of active optical devices with the potential to correct refractive error and ocular aberrations in real-time (e.g. wavefront correction systems and ‘adaptive optics’ systems), it is contemplated that the design approaches of this invention may also be incorporated in these devices.

Many modifications, variations, and other embodiments of the invention will come to the mind of one skilled in the art to which this invention pertains having the benefit of the teachings presented in the foregoing descriptions. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A method for controlling optical aberrations to alter modulation transfer functions comprising the steps of:
providing an ocular system comprising a predetermined aberration-controlled design to reposition medium- and high-spatial frequency peaks relative to one another; said repositioning of peaks producing substantially corrective stimuli; and
providing the substantially corrective stimuli to an eye to reduce accommodative lag, wherein the repositioning is effected while substantially simultaneously providing clear visual images.

2. The method according to claim 1 wherein the stimuli is provided substantially continuously.

3. The method according to claim 1, wherein the ocular system exhibits substantial centration relative to the eye.

4. The method according to claim 1, wherein the predetermined design provides a negative spherical aberration.

5. The method according to claim 1, wherein the step of repositioning the medium- and high-spatial frequency peaks further comprises repositioning the medium-spatial frequency peak to a point located a distance from the cornea of the eye and towards the retina greater than the distance from the cornea to the high-spatial frequency peak.

6. The method according to claim 5, wherein, for an eye exhibiting myopia, axial elongation is reduced.

7. The method according to claim 5, wherein, for an eye exhibiting myopia, the myopia progression is reduced.

8. The method according to claim 1, wherein the step of repositioning the medium- and high-spatial frequency peaks further comprises repositioning the high-spatial frequency peak to a point located a distance from the cornea of the eye and towards the retina greater than the distance from the cornea to the medium-spatial frequency peak.

9. The method according to claim 8, wherein, for an eye exhibiting hypermetropia, the hypermetropia is abated.

10. The method according to claim 1, wherein the ocular system is selected from the group consisting of contact lenses, orthokeratology lenses, on-lays, in-lays, anterior chamber lenses, intra-ocular lenses, corneal sculpting, and combinations thereof.

11. The method according to claim 10, wherein the contact lenses are selected from the group consisting of extended wear contact lenses and continuous wear contact lenses.

12. The method according to claim 1, wherein the repositioning of the medium- and high-spatial frequency peaks relative to one another is accomplished by methods selected from the group consisting of orthokeratology and refractive corneal sculpting.

13. The method according to claim 12, wherein refractive cornea sculpting method is selected from the group consisting of, epikeratophakia, thermokeratoplasty, LASIK surgery, LASEK surgery, and PRK surgery.

14. An ocular system comprising a predetermined corrective factor to reposition medium- and high-spatial frequency peaks relative to one another to produce a substantially corrective stimuli to an eye to alter accommodative lag, wherein the repositioning is effected while substantially simultaneously providing clear visual images.

15. The system according to claim 14, wherein the stimuli is provided substantially continuously.

16. The system according to claim 14, wherein the predetermined corrective factor provides a negative spherical aberration.

17. The system according to claim 14, wherein the ocular system exhibits substantial centration relative to the eye.

18. The system according to claim 14, wherein the medium-spatial frequency peak is repositioned to a point located a distance from the cornea of the eye and towards the retina greater than the distance from the cornea to the high-spatial frequency peak.

19. The system according to claim 14, wherein the high-spatial frequency peak is repositioned to a point located a distance from the cornea of the eye and towards the retina greater than the distance from the cornea to the medium-spatial frequency peak.

20. The system according to claim 14, wherein the ocular system comprises a device selected from the group consisting of contact lenses, orthokeratology lenses, on-lays, in-lays, anterior chamber lenses and intra-ocular lenses.

21. The system according to claim 20, wherein the contact lenses are selected from the group consisting of extended wear contact lenses and continuous wear contact lenses.

22. The system according to claim 14, wherein the predetermined corrective factor is introduced to the system via an orthokeratology method.

23. The system according to claim 14, wherein the predetermined corrective factor is introduced to the system via a corneal sculpting method.

24. The system according to claim 23, wherein the corneal sculpting method is selected from the group consisting of epikeratophakia, thermokeratoplasty, LASIK surgery, LASEK surgery, and PRK surgery.

25. An ocular device comprising a predetermined prescriptive strength and predetermined aberrations to predictably reposition high- and medium-spatial frequency peaks relative to one another and deliver predetermined stimuli to an eye, wherein the repositioning is effected while substantially simultaneously providing clear visual images.

26. The device according to claim 25, wherein the device causes the medium spatial frequency peak to be repositioned to a point located a distance from the cornea of the eye and towards the retina greater than the distance from the cornea to the high spatial frequency peak.

27. The device according to claim 25, wherein the device causes the high spatial frequency peak to be repositioned to a point located a distance from the cornea of the eye and towards the retina greater than the distance from the cornea to the medium spatial frequency peak.

28. The device according to claim 25, wherein the stimuli is provided to the eye substantially continuously.

29. The device according to claim 25, wherein the device is selected from the group consisting of contact lenses, orthokeratology lenses, on-lays, in-lays, anterior chamber lenses and intra-ocular lenses.

30. The device according to claim 29, wherein the contact lenses are 5 selected from the group consisting of extended wear contact lenses and continuous wear contact lenses.

31. The device according to claim 25, wherein the aberrations are controlled through the use of optical design features selected from the group consisting of conic sections, polynomials, splines, Bezier curves and surfaces, Fourier series syntheses, Zernike polynomials, sagittal height descriptions and look-up tables, gradient refractive index profiling, Fresnel optical components, diffractive optical components, holographic optical components and combinations thereof.
